(19) United States
(12) Patent Application Publication
(10) Pub. No.: US 2025/0213394 A1
(43) Pub. Date: Jul. 3, 2025

(54) SYSTEM, DEVICE AND METHOD FOR GENERATING PLANNING DATASETS FOR THE TISSUE TREATMENT OF A PATIENT'S RETINA

(71) Applicant: OD-OS GMBH, Teltow (DE)

(72) Inventors: Annemarie Hoff, Potsdam (DE); Arnd Rose, Stahnsdorf (DE); Kay-Uwe Amthor, Teltow (DE)

(21) Appl. No.: 18/850,800

(22) PCT Filed: Mar. 24, 2023
(86) PCT No.: PCT/EP2023/057715

§ 371 Date: Sep. 25, 2024

(30) Foreign Application Priority Data
Mar. 25, 2022 (EP) 22164535.1

(51) Int. Cl. A61F 9/008; G06T 7/00; G16H 10/60; G16H 20/00; G16H 30/20

(52) U.S. Cl. CPC A61F 9/008; G06T 7/0012; G16H 10/60; G16H 20/00; G16H 30/20; A61F 2009/00878; G06T 2207/30041

(57) ABSTRACT

The invention relates to a system for creating planning data sets (34, 35, 36, 37) for the tissue treatment of the retina (16) of the eye (4) of a patient by means of a laser (2), with a camera (6, 7) which is configured to continuously capture images of the retina, with an image processing device (19) which is configured to generate and provide current structured image data of the retina from images captured by the camera, with a personal data acquisition device (21, 22), which is configured to acquire and/or provide personal data of a patient, and with a processing device (20), which is designed to assign planning datasets for the tissue treatment to the current structured image data of the retina and to the patient's personal data, wherein specifications, in particular patterns or rules, for the assignment of planning data to image data and patient's personal data are stored in the processing device.

The invention is in the field of medical technology and can be used particularly advantageously in the field of ophthalmology and in conjunction with certain ophthalmoscopes.

In many cases, laser treatment devices are used to treat a patient's retina, which enable tissue treatment using a laser beam. The combined use of an ophthalmoscope and a laser treatment device is particularly advantageous in order to enable simultaneous and coordinated visualization of the retina during treatment.

The basic function of an ophthalmoscope is to provide an examining person with an image of an eye or parts of an eye. In particular, an ophthalmoscope that provides an image of the retina of an eye can be combined with a device for laser treatment of the retina if, for example, the same optical imaging device is used at least partially for guiding the laser beam as for visualizing the retina. In this case, the beam paths for imaging and laser treatment can, for example, be branched over part of their length by beam splitter devices and run together through the same optical elements over another part. This makes it possible to observe the retina at the same time as the laser treatment, which would be much more difficult for reasons of space alone if separate imaging devices were used for observation and treatment.

European patent application EP 1 875 857 A1 discloses an ophthalmoscope with imaging optics and an illumination device as well as an optical sensor with which images of the retina of an eye can be captured. Such an ophthalmoscope can also be used for planning laser treatment by a surgeon.

Against the background of the prior art, the present invention is based on the task of facilitating the creation of treatment plans for the tissue treatment of a retina by means of a laser device and of systematically standardizing and improving the planning data.

According to the invention, the object is solved by a system with the features of claim 1. The dependent claims present advantageous implementations of such a system. To solve the problem, the invention also relates to a device and a method for creating planning datasets, which are set out together with advantageous embodiments in further patent claims.

Accordingly, the invention relates to a system for generating planning datasets for the tissue treatment of the retina of a patient's eye using a laser,
with a camera that is configured to continuously capture images of the retina,
with an image processing device which is configured to generate and provide current structured image data of the retina from images captured by the camera,
with a personal data acquisition device which is configured to acquired and/or provide personal data of a patient,
   and with a processing device which is configured to assign planning datasets for tissue treatment to the current structured image data of the retina and the patient's personal data, wherein specifications, in particular patterns and rules, for the assignment of planning data to image data and personal data of patients are stored in the processing device.

In addition, it may be advantageous for the system to have a modification device, which may also be referred to as a correction device and which enables manual modification of a planning dataset. Such manual modifications can be made during or especially after the creation of a planning dataset by a skilled person based on their experience or additional knowledge.

The system according to the invention is configured to support the creation of a treatment plan, which is created in the form of a planning file or planning data, before the start of treatment of a patient's retina. A prerequisite for this is that the system has a camera or generally an imaging system that is configured to continuously capture images of the retina. Such a camera can be designed as an optical color, infrared or black-and-white camera or as a scanning system or also have various filter options and enables the acquisition of current images of the retina before and/or during the generation of planning data. This can be important, for example, in order to be able to compare other acquired or currently determined retinal data with the currently acquired status and positioning of the retina in the system. In the context of the present invention, structured graphical or image data is to be understood as all preparations of pure pixel data and the data derived from these or from the pixel data, in particular so-called vector-oriented image data. For example, the position and size of blood vessels, the position of the optic nerve and the position, size and type of drusen, namely soft drusen, hard drusen and reticular pseudodrusen (RPD), can be provided as structured image data and taken into account when creating planning datasets.

If, for example, patient personal data is used, this may be in the form of a diagnosis or indication or of images of the patient's retina previously taken by the system itself or by another imaging device. These can then be compared with a current recorded image in the system. Personal data can also be data from a previous treatment carried out on the patient or data on a treatment status. In principle, personal data can be all patient data already available in written or electronically, in particular digitally, stored form before the start of treatment or before the start of the creation of the planning dataset. These can be transmitted to the system via electronic interfaces or entered manually. The personal data may, for example, be available in electronic form in a more comprehensive system, of which the system for creating planning datasets according to the invention is a subsystem or subsystem.

An advantage of a camera that captures actual images of the retina arises when the system for generating planning data is also combined with a treatment device that has a treatment laser that is adjusted relative to or aligned with the camera to perform the treatment using the planning data. The structuring of the planning data is explained in detail below.

Furthermore, an image processing device is provided which generates structured image data of the retina from the currently captured image data of the camera and makes it available for planning. Structured image data is, for example, data that contains or represents certain objects recognized in the images by the image processing or also properties of the image or the retina or properties of image regions or parts of the retina. For example, the structured image data can describe brightness or color distributions, intensity distributions on an infrared image, the position, shape and/or gradients of objects or object boundaries. The representation of such structures in the form of data can be diverse and contain, for example, pure image data, but also vectorial representations, vectors, matrices or other types of abstracted representations. For example, the structured data should describe the shape, position and possibly other parameters of blood vessels, nerve nodes and general abnormalities on the retina in a clear form, for example vector-oriented or with metadata relating to geometric shapes.

The position of objects that are currently recognized on the retina, such as blood vessels, the optic nerve or drusen, can and should be taken into account when creating a treatment plan and generating corresponding planning data.

The extent to which such currently acquired structured image data is taken into account may depend, among other things, on the patient's personal data. As explained above, the personal data refers to data relating to the patient that already exists and is available when the system is applied to the patient.

The system may provide for certain elements of the system to be locally available where the patient is being treated, while other elements may be located elsewhere or be difficult to localize. For example, the image processing device or the processing device for generating planning data or parts of this device, such as storage devices, can be located at a different place than the treatment laser. Data processing equipment may, for example, be located in larger computers or data processing systems than are available at the laser treatment site. If the processing device needs access to a large amount of stored data, both the processing devices and the storage devices can be distributed over several computers or located in a larger, central computer system. For example, it may be useful to store treatment data of many patients in the form of a database centrally or in a distributed computer system in order to have as much data as possible available and accessible to the system when interpreting individual data of a single patient or for training a learning system.

Individual parts or modules of the system, such as the personal data acquisition device, a measurement data acquisition device or a modification device for manual correction of planning data or a device for entering data describing the success of treatment, can also be installed as an application on a mobile device, for example. The data formats of the transmission of input data to the mobile device and the output from the mobile device to a stationary part of the system, which also contains the camera and/or the processing device, are then carried out via interfaces in which both the data formats and the data transmission paths, such as hardware connections, data bus or radio interface, are clearly defined in order to obtain an integrated system.

One embodiment of such a system can, for example, provide for one of several predefined patient categories to be assigned to a patient on the basis of personal data and/or currently acquired data and for specifications of the processing device (20), in particular patterns and rules, for the assignment of planning data to currently acquired data and personal data to be determined or influenced by the respectively assigned patient category.

The patient category that is assigned to the respective patient can, for example, depend on stored personal data, such as age, certain physiological parameters of the patient, such as weight, type and strength of skin pigmentation, hair color, pigmentation of the iris, medical history and the type, intensity and success of pre-treatments that have been carried out and/or on currently measured or acquired data, such as the structured image data of the retina or currently acquired physiological data and/or parameters of the patient, which may either be measured in or on the eye or may also be independent of parameters of the eye, such as the patient's age, eye color, hair color, skin color, strength of skin pigmentation, physique and current state of health.

In one embodiment, for example, only two patient categories can be provided, which are determined by a single acquired parameter, for example patients younger than 50 years or older or light-skinned and dark-skinned patients. However, more than two, for example 3, more than 3, more than 5 or even more than 10 categories can also be provided, whose respective assignment to a patient also depends on more than one parameter, for example on at least two or at least three parameters.

Depending on the patient category assigned to the individual patient, predefined planning datasets can be assigned in some cases. In other cases, the planning datasets can still be created individually using saved specifications, in particular patterns and rules, but the patterns and rules used may depend on the assigned patient category. This allows the specifications to be made less complex.

It is also conceivable, depending on the patient category assigned to a patient, to limit certain categories of planning data, such as the laser parameters or target values for measurement data, for example the target data that represents the current maximum permissible temperature of the retina at the treatment site during treatment, so that only a subset of possible parameters is available for the planning datasets. In many cases, this simplifies and accelerates the creation of a planning dataset after a specific patient category has been assigned to an individual patient.

However, it is also conceivable to create certain patient categories for patients that are identified as problem categories and require special planning, for example particularly careful or small-scale planning, or exclude or make impossible the automatic or semi-automatic creation of a planning dataset. In this case, an assigned patient category could also refer to fully expert-based treatment planning.

For example, a patient category in which patients with heavily pigmented skin and/or heavily pigmented retina and/or a dark iris color are classified could be linked to the rule that certain laser power densities must not be exceeded.

Certain patient categories relating to pre-existing conditions may, for example, be linked to the condition that certain retinal temperatures, which are monitored by acquiring certain measurement data during treatment, must not be exceeded. Alternatively, the target data corresponding to the desired temperature of the retina at the treatment site during treatment could be selected depending on the patient category determined by previous diseases or pre-treatments. Similarly, the selection of location data for laser treatment and the selection of location data for regions excluded from laser treatment may also depend on the patient category.

A further advantageous embodiment of such a system can provide that the personal data acquisition device is configured to acquire and provide diagnostic data and/or indication data and/or data of a pre-treatment carried out and/or previously known physiological data of the patient and/or already existing images of the patient's retina stored outside the system.

Such personal data can, for example, be in the form of an electronic file, can be entered manually or can be read in, for example a diagnosis in text or image form or an indication that already provides information on a recommended treatment. Such information may already contain data that differentiates certain locations or regions of the retina and is location-dependent from the respective location on the retina, such as the exact location of sites or regions to be treated. However, such information can also be location-independent and describe the patient's general condition, their eyes or other characteristics, such as physiological data. Such data can be in the form of older images of the retina of different types, for example color images, black and white images, infrared images, fluorescence images or tomography images, or in the form of age information, skin color or gender of the patient, an identity of the patient or information about pre-treatments which may have taken place with or without the involvement of the system according to the invention.

Furthermore, in one embodiment, such a system can be designed in such a way that a measurement data acquisition device is provided which has one or more sensor devices for acquiring and providing physiological data of the patient, in particular data which depend on the respective location on the retina and/or data which do not depend on a location on the retina, and that the processing device is configured to take the acquired physiological data of the patient into account when assigning planning data.

Such measurement data acquisition devices can basically be any type of device that enables the current acquisition of additional information about the patient and his physiological data. This includes sensor devices such as cameras that record other properties than the camera mentioned at the beginning, for example, other imaging processes or also use similar or the same imaging process as the camera mentioned at the beginning for recording the retina. Images of the retina captured with such cameras then provide data that is dependent on the respective location of the retina. For example, a sensor device can be used to measure the temperature or temperature distribution on the retina. Using images of the retina, for example, algorithms can also be used to detect retinal disease, which can be taken into account for the creation of planning data. A device for creating an OCT image or a device for reflectometry can also be provided. With a camera, in particular also with the same camera that is configured to continuously capture images of the retina, reactions of the retina to titration shots, i.e. experimental applications of the treatment laser, can also be captured in order to be able to plan and control the laser parameters based on these reactions or at least to be able to take these reactions into account when generating planning data. Sensor devices that measure other physiological characteristics of the patient are also conceivable, such as skin color and/or brightness, skin thickness, hair color, color and/or color distribution of the iris, blood pressure, various blood values or similar. These sensor devices can also include a camera or optical sensors, for example.

A special implementation of the system can, for example, also provide for the above-mentioned measurement data acquisition devices to continue to be operated to acquire data even after an initial generation of planning data for a laser treatment and possibly also after a manual correction of planning data during the actual laser treatment. For example, the temperature of the retina or temperature distribution on the retina can be continuously acquired by such a measuring device even during treatment or during breaks in treatment. The measurement data obtained in this way may make it necessary or advantageous to change the planning data. It may therefore be necessary for the system to generate a further, corrected planning file based on the measurement data determined after the initial creation of a planning dataset and to use this as the basis for further treatment.

The system can also be further formed, for example, by a display module or a display device which effects the graphic representation of a planning dataset within an image of the retina currently captured by the camera. In this way, the planning data can be visualized together with a graphical representation of an image of the retina, so that, for example, the consideration of the structured image data can be assessed immediately by making it visible whether, for example, certain sensitive regions of the retina, such as blood vessels or nerve nodes, are excluded from treatment or whether certain regions already visible in the current image and classified as requiring treatment are appropriately considered in the treatment plan.

In addition, if a treatment device is integrated into the system and if the current camera image is synchronized with the steering device for a treatment laser, the planning dataset can also be synchronized directly with the steering device.

The system can also be further enhanced by a tracking device that causes the display of the planning dataset to be tracked when the image currently captured by the camera is moved, in particular when it is shifted or rotated. Such a shift in the image can occur, for example, if the patient moves in relation to the camera or if the eyes move, and in such a case the tracking device ensures that the display of the planning data continues to match the current camera image displayed. If a treatment device is integrated into the system, such tracking can also ensure that the steering device for a treatment laser remains aligned as planned.

In addition, in a system of the type mentioned, it can be provided that the processing device has a storage device which is configured to store personal data of patients and planning datasets assigned to them, and in particular additionally to store physiological data associated with the aforementioned data and acquired by a measurement data acquisition device and/or to store manual modifications made to the planning datasets.

This enables the system to compare the assignment of a current planning dataset to a patient's personal data with previous assignments for similar or different personal data. This can be used to carry out a plausibility analysis, for example. If planning changes have been made manually at a later date in earlier cases, these can also be taken into account and anticipated during planning, for example. In addition, if acquired physiological data is also stored, this can also be taken into account when comparing and reconciling the planning file to be created with previous plans. Accordingly, the system according to the invention can be designed as an expert system or as a learning system.

Furthermore, in such a system it may be provided that the processing device has a storage device which is configured to store personal data of patients as well as structured image data and/or images of the patient's retina generated with the system and planning datasets assigned to these, in particular target values for measurement data, inter alia, and in particular additionally to store physiological data associated with the aforementioned data and acquired by a measurement data acquisition device and/or for storing manual modifications made to the planning datasets.

This embodiment of the system makes it possible to compare the assignment of a current planning dataset to the personal data of a patient and the structured image data with previous assignments for similar or differing personal data and structured image data that are also stored. A plausibility analysis can also be carried out with this data. If planning changes have been made manually at a later date in earlier cases, these can also be taken into account and anticipated during planning, for example. In addition, if acquired physiological data is also stored in the expert system created in this way, it can also be taken into account when comparing and reconciling the planning file to be created with previous plans.

A further development of the system can be provided, for example, by an inspection device, which acquires and provides data on the effects of the treatment after the retina has been treated.

The treatment of the retina may include laser treatment, but may also include additional treatment steps with or without the use of a laser. The check can be carried out immediately during treatment or after completion of treatment, in particular also a certain minimum time after treatment. The inspection device can comprise a device for carrying out an imaging process, for example also using the camera mentioned at the beginning to capture current images of the retina. The inspection device can also include an input device for manually entering data. It may be intended that only binary information such as “treatment successful” or “treatment unsuccessful” is acquired or an assessment by the patient, such as “have pain, yes/no” or a further assessment, for example, which parameters of the treatment, such as the laser parameters, selected geometric treatment patterns, regions intended for treatment or regions excluded from treatment, were suboptimal in the opinion of an assessor. For this purpose, certain categories of assessment can be specified, such as an evaluation scheme. Such an assessment can also be included in the expert systems described above, stored together with the saved planning datasets and taken into account when comparing current planning data with saved planning data.

The planning datasets generated by the system can contain planning data from one, two, three, four or five of the following categories in an implementation of the system: Laser parameters, location data for laser treatment, treatment patterns, location data for regions excluded from laser treatment, and target values for measured values acquired during a subsequent treatment by the measured value acquisition device or otherwise. Such target values can be, for example, an average target temperature or a target temperature depending on the location on the retina, which is continuously acquired during the laser treatment and which should, for example, be reached, maintained or not exceeded. The other treatment parameters can then be dynamically adjusted during treatment so that the specified target values are reached or not exceeded. For example, parameters for a rule are thus specified in the planning datasets. In addition, report templates for treatment can also be generated as a further category of planning data, i.e. forms and the selection and presentation of the data and parameters to be reported.

The invention can be further advantageously realized in that the processing device is designed as a self-learning device in which the assignment function, which assigns planning data to the personal data and in particular to the structured image data and in particular to the measured physiological data of a patient, is trained in that at least data from at least one of the following categories is, on the one hand, specified for it as training data for each patient: personal data, structured image data, measured physiological data and, on the other hand, assigned planning data, in particular target values for measurement data, as well as manual modifications to planning data and, in particular, data on an effect of the treatment acquired after the planned treatment has been carried out.

The planning datasets generated by the system can contain planning data from one, two, three, four or five of the following categories: Laser parameters, location data for laser treatment, treatment patterns, location data for regions excluded from laser treatment, target values for measurement data. The laser parameters can include, for example, the laser power, wavelength, pulse duration, repetition rate of the pulses and the total number of laser pulses as well as the size of the light spot of the laser on the retina and each of these parameters or all conceivable sub-combinations. These laser parameters can depend on the location or region on the retina. The location data of the laser treatment can include one or more geometric regions on the retina. The regions of the retina excluded from treatment may also include one or more geometric regions of the retina. The treatment patterns can include geometric patterns such as circles, ellipses, rectangles, triangles or any free-form shapes in which the laser treatment is to take place with constant laser parameters, for example, or in which one of the laser parameters remains the same. These treatment patterns can also be areas that are processed, i.e. irradiated, independently by the treatment laser after a start impulse from the surgeon. The treatment patterns can also simply comprise geometric regions that the laser processes in one go, for example point by point according to a point pattern, without the surgeon having to control each individual point. Only one starting point can be controlled manually or automatically, and the rest of a treatment pattern can then be automatically controlled by the laser control system. The density of the points to be addressed can also be one of the parameters in the “Treatment patterns” category. Planning data can also be target values for measurement data acquired during the treatment, such as a temperature to be reached or not exceeded on the retina during the treatment, which can be specified as an overall average value or location-dependent and which is continuously acquired/measured during the treatment. The treatment system is then controlled in a way that the target values are met.

Continuously detectable optical variables, such as a change in reflection detected by a camera or a hyperspectral sensor, discoloration/spectral change or change in brightness of the respective treated regions can also be specified as target values.

Each of these mentioned categories of planning data, or more precisely the data of each of the mentioned categories, can be manually changed by a person, in particular by the respective operator, if necessary with restrictions as explained above. The operator can compare the planning data generated by the system in the first attempt with the image data currently captured by the camera or the structured data as well as with his experience and modify it according to his assessment.

The regions to be excluded from treatment can play a special role here. Although in many cases they can be thought of as complementary to the location data for the laser treatment, in many cases it is less error-prone to specify the regions of the retina to be excluded from the treatment than the location data for the treatment, especially if, for example, the retina is to be treated uniformly except for a few regions to be excluded. In addition, the regions to be excluded can be very relevant to safety, as they also include regions where laser treatment can cause damage to health. It may therefore also be possible, for example, to define the regions to be excluded from treatment according to the planning data generated by the system in such a way that they cannot be changed manually or that certain obstacles must be overcome or authorizations must be proven before they can be changed.

The system can be designed as a self-learning system that uses the above-mentioned data provided to it to continuously improve the assignment algorithm that generates planning data. The system can have a neural network that is trained, or it can have an analytical, statistical or parametric learning algorithm, be static, guided learning or self-learning. The assignment algorithm can also have analytical elements that cannot be changed by the learning process and are specified with expert knowledge, such as certain conditions that the laser parameters must fulfill, for example power limits or regulations that the exclusions of treatment locations must follow.

For the learning process based on the available treatment data, which can be carried out regularly when the available treatment data is expanded and increased, the elements of the learning process can be ordered and weighted, for example. For example, the data based on the treatment successes determined after the laser treatment with the monitoring device can be given a higher weighting than the manual corrections of planning data made by treating persons. It is also possible, for example, to introduce conditions that must be met for treatment data to be made available to the learning system or the learning algorithm. For example, one such condition may be that certain manual parameter changes, such as the laser parameters, must have been made within a defined range in a certain minimum number of cases or by a minimum number of treating persons or at a minimum number of treatment locations in order for these changes to be provided to the learning system as training data.

In addition to a system of the type described above, the invention also relates to a device for generating planning datasets for the tissue treatment of the retina of an eye by means of a laser beam,
with a camera that is configured to continuously capture images of the retina,
with an image processing device that generates and provides current structured image data of the retina from images captured by the camera,
with a personal data acquisition device which is configured to acquired and/or provide personal data of a patient,
   and with a processing device which is configured to assign planning datasets to the current structured image data of the retina and the patient's personal data, whereby specifications, in particular patterns and rules, for the assignment of planning data to structured image data and personal data of patients are stored in the processing device. The device can advantageously also have a modification device that enables the manual modification of a planning dataset.

In addition to a system and an apparatus, the invention also relates to a method for generating planning datasets for the tissue treatment of the retina of a patient's eye by means of a laser beam,
in which at least one image of the retina is captured with a camera,
in which an image processing device is used to generate and provide current structured image data of the retina from images captured by the camera,
in which a patient's personal data is acquired and/or provided as part of a personal data acquisition device,
and in which planning datasets are assigned by a processing device to the current structured image data of the retina and the previously known personal data of the patient on the basis of specifications, in particular patterns and rules, which are stored in the processing device.

The method can be advantageously designed, for example, so that assigned planning datasets are changed manually after assignment.

Furthermore, the method can be advantageously realized in that physiological data of the patient are acquired and provided, in particular with a measurement data acquisition device, and taken into account in the assignment of planning datasets by the processing device. The physiological data can also be acquired and entered by a person, especially if it concerns easily recognizable characteristics of the patient, such as hair or iris color.

The method can also be advantageously realized in that the processing device is designed as a self-learning device and the assignment function, which assigns planning data to the personal data and the structured image data and, in particular, physiological data of a patient measured by a measurement data acquisition device, is trained in that the personal data, structured image data, assigned planning data, data of the manual planning data changes and, in particular, measured physiological data of a patient are assigned to it for a plurality of patients or acquired physiological data and/or data on the effect of the treatment acquired after the planned treatment has been carried out.

In a first step, it may be provided that one of several predefined patient categories is assigned to a patient on the basis of personal data and/or currently acquired data and that specifications of the processing device, in particular patterns and rules, for the assignment of planning data to currently acquired data and personal data are determined or influenced by the respective assigned patient category.

As with the system described above, the method can be designed in such a way that planning data from one, two, three, four or five of the following categories are generated as planning datasets: Laser parameters, location data for laser treatment, treatment patterns, location data for regions excluded from laser treatment, target values for measurement data.

The different types, appearances and forms of presentation of the individual types of planning data have already been described in detail above.

The invention may further relate to a method of operating a system and/or device of the type described above, in which a planning dataset is created and then a treatment is carried out in accordance with the planning dataset in a separate treatment device or a treatment device integrated into the system or device by means of a treatment laser.

The success of the treatment can then be assessed using a monitoring device, for example, and data representing the success can be fed to the system in digital form to enable the system to learn.

The invention is shown by means of embodiment examples in figures and explained below. In the figures:

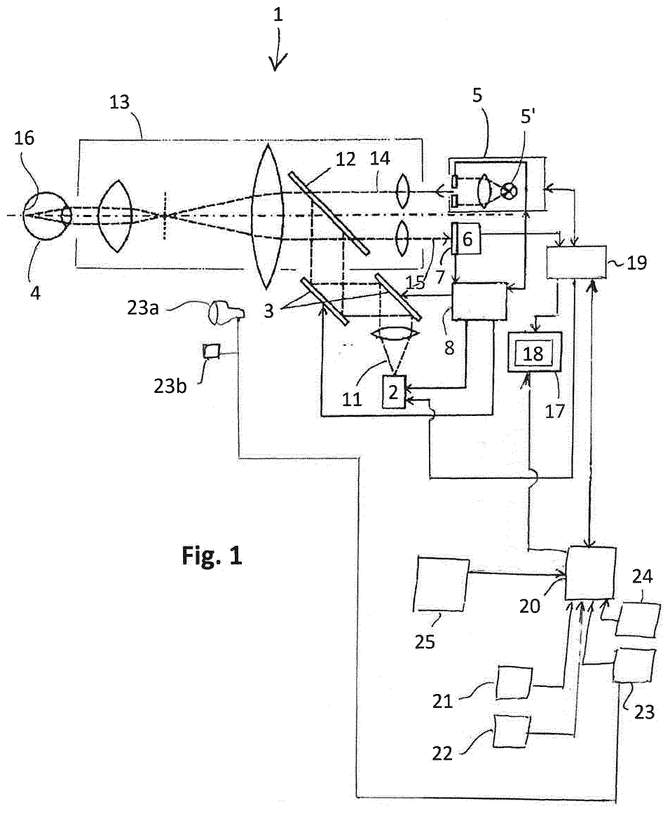

FIG. 1 shows essential parts of the device according to the invention and the system with a camera integrated in an ophthalmoscope and combined with a treatment laser, an image processing device, a processing device for generating planning data and some further data acquisition and input devices,

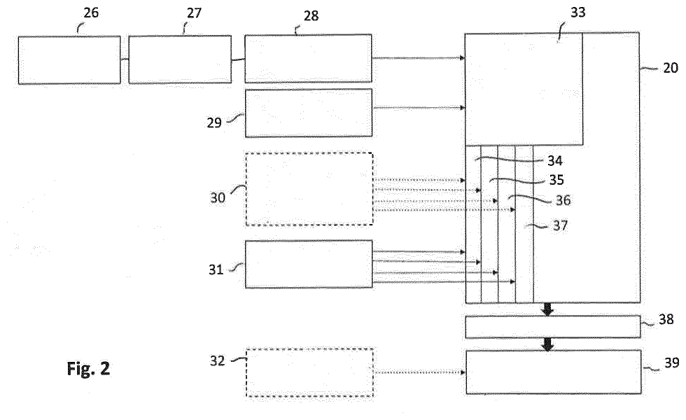

FIG. 2 shows schematic of a system for creating planning datasets,

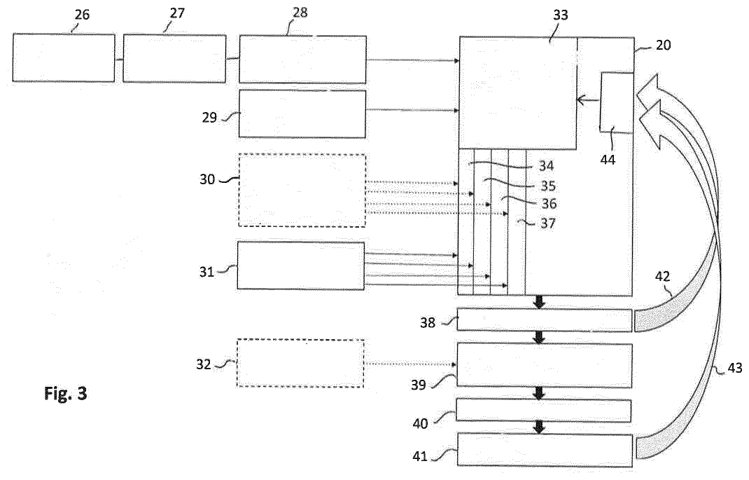

FIG. 3 shows schematic of a system for creating planning datasets with a learning algorithm, and

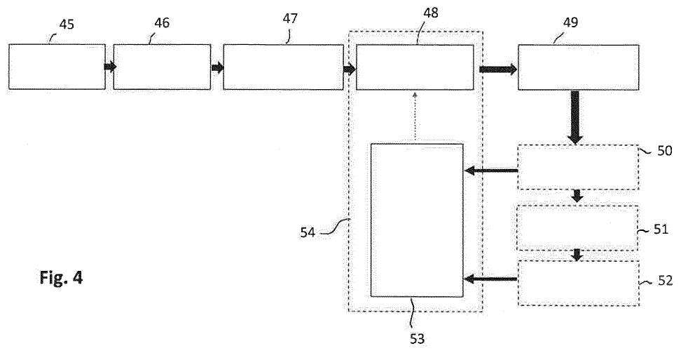

FIG. 4 shows an exemplary flow chart for the method according to the invention.

FIG. 1 shows at least partially a device and a system according to the invention with a schematized ophthalmoscope 1, which can be used in the system for carrying out the method according to the invention and combines the functions of an ophthalmoscope with a camera with functions of laser treatment of the retina.

The device has an illumination device 5 with a radiation source 5′, which is configured to direct an illumination beam 14 onto the eye 4 and the retina 16 of a patient. This allows the retina 16 to be suitably illuminated for capturing an image or a camera image. The lighting can, for example, be equipped with a light-emitting diode or an infrared diode as a light source, or with a different type of light source that provides a desired wavelength spectrum. The light source can also be a UV light source, for example.

The ophthalmoscope also has a camera 6 with an image sensor labeled 7. The sensor can be a CCD sensor, for example. Instead of the camera 6, any other type of device can also be provided which, for example as a scanning device, is suitable for detecting radiation that reaches a sensor from the retina and generating an image of the retina. The goal of operating the illumination device 5 and the camera 6 or an equivalent device is to obtain the most accurate, spatially resolved measurement data possible from the retina 16 by capturing an image of reflected radiation and thus to acquire the properties of the entire retina or of target regions to be treated or to be able to determine them from acquired data.

The illumination beam 14 and the reflected radiation 15 are suitably collimated or focused by a suitable optical system 13 with mirrors and lenses in a manner known per se. The optical system 13 also has a beam splitter 12, which makes it possible to direct a laser beam 11 from the treatment laser 2 onto the retina 16. Instead of a beam splitter, different beams of the treatment laser and the illumination and/or image acquisition can also be guided parallel to each other at a certain distance.

A control unit 8 can be provided to control the laser 2, which on the one hand controls the lighting device 5, for example triggers it, and on the other hand directly controls the laser 2 on the basis of a camera image from the camera 6. The control unit 8 can also control deflecting mirrors 3, which direct the beam path of the treatment beam 11 and thus enable the treatment of individual target regions on the retina 16. In the system according to the invention, an image processing device 19 is also provided, which permits digital processing of images/camera images from the camera 6 and determines structured data from the image data, which is present, for example, in the form of pixel data, and which can be provided, for example, in the form of a vector graphic or in another structured form. The image data and/or structured image data can also be made available to the control unit 8. The image processing device 19 also provides the data for the display device 17, which has a screen 18 on which, for example, an image of the retina is displayed. In the display device 17, in addition to the image data of the camera 6 and/or structured image data of the image processing device 19, planning data can also be processed and displayed together with the image data in numerical or graphical form. For example, treatment regions can be marked and/or treatment patterns or laser parameters can be displayed in the current image data, i.e. in a live view of the retina. A tracking device is also provided in the display device or connected to it, which, in the event of movement of the camera image, i.e. movement of structures shown in the camera image, also moves the elements of the planning data that are assigned to the image data.

FIG. 1 also shows the processing device 20 as part of the device and the system, which generates planning files or planning data for the laser treatment and uses at least personal data from a personal data memory 21 or personal data of the patient to be treated entered by means of a first input device 22 as well as structured camera images. In addition, the processing device can use measurement data from a measurement data acquisition device 23, as well as correction data or change data that can be entered by an operator after the first planning data generation by means of the second input device 24. The measurement data device 23 may, for example, comprise sensors 23b such as color sensors, a camera 23a for objectively detecting the patient's skin or hair color or a camera directed at the iris of the eye to be treated for detecting the iris color, or may be connected to such sensors. The measurement data device can also use the camera 6 for measurements and point it at the iris, for example, or use this camera to record data other than the image data of the retina. This data acquisition can, for example, be controlled directly by the measurement data acquisition device 23 or also mediated by the image processing device 19.

The processing device can send planning data by means of the image processing device 19 or directly to the display device/display module 17, where the planning data can be displayed together with current images of the retina on a screen 18. Some of the planning data can be displayed numerically, but at least some of it can also be displayed directly graphically in an image of the retina.

After the laser treatment, the success of the treatment can be assessed and reported to the processing device 20 by means of a success control device/monitoring device 25, which, among other things, can receive current images of the retina 16 from the camera 6. The processing device 20 has a learning algorithm or a self-learning device that improves the assignment algorithm for mapping planning data to output data using training data.

According to the state of the art, the type and intensity of the laser treatment, i.e. the strength and/or duration as well as the temporal and local distribution of the laser pulses, was previously carried out by a surgeon according to his own assessment after evaluating an image of the retina. Titration pulses were often first directed at the retina and their effect assessed in order to scale the laser intensity.

The largely autonomous, automated assignment of planning data to personal data and image data and possibly other data in accordance with the invention makes it possible to assign planning data to each target region on the retina in an objectified, transparent and comprehensible manner, even without titration shots, wherein the planning data may include different categories of data, such as laser parameters, treatment locations or regions, locations or regions excluded from treatment, and treatment patterns.

FIG. 2 shows a system according to the invention with an image acquisition device 26 of the retina and an image processing device 27 which supplies structured image data 28 to the processing device 20. The structured data can contain, for example, recognized objects such as nerve nodes, blood vessels and recognized diseased tissue regions as well as data on the color and color intensity of the retina and other graphically recognizable properties such as contrasts, brightness and color distributions and other comparable data. Color intensity and brightness values can be used, for example, to determine the pigmentation intensity of the retina, which can be decisive for the absorption of laser light of certain wavelengths and thus for the necessary laser intensity for a treatment. In the context of the present invention, structured graphical data should be understood to mean all preparations of pure pixel data and data derived from these.

In the figure, 29 indicates the patient's personal data that is provided to the processing device 20. This can be done by electronic transfer of data or by manual entry. Potentially, the processing device 20 can also be supplied with measurement data from a sensor system or measurement data acquisition device, which is labeled 30 in FIG. 2. This data can be retinal location-dependent data, such as a currently measured temperature distribution on the retina, or data obtained from OCT (optical coherence tomography), which can indicate edema, for example, or color and/or brightness measurements of the retina. However, they can also be other measured or estimated variables that are independent of a retinal location, such as the patient's skin, hair or iris color, which can be measured or estimated and entered manually.

The processing device 20 contains an element 33 in the form of an assignment device, which contains patterns and rules for the assignment of planning data to input data (current image data or structured image data, personal data, sensor-detected data).

In the example shown, the assignment device outputs planning data of the categories laser parameters 34, location data 35 for the treatment, treatment patterns 36 for the treatment and data of the locations 37 excluded for the treatment.

After the planning data has been output and, if necessary, graphically displayed, an operator can manually change the planning data of one, some or all categories using the element 31 by means of an input device. The modified planning data is then usually used as the basis for laser treatment. The modified planning data then finally form the planning data 38 for the laser treatment, which can also be displayed in the graphic representation 39, for example together with current images of the retina. The system shown in FIG. 2 can thus function in a static manner if the patterns and rules of the assignment device 33 stored in the processing device 20 are sufficiently good and valid. Such patterns and rules can, for example, be made available as part of an expert system and also regularly updated.

FIG. 3 shows a system in which the patterns and rules according to which the assignment device 33 performs the assignment or generation of planning data are continuously corrected and/or improved in the processing device 20 as part of a learning algorithm.

For example, the planning data for the treatment resulting after manual corrections 31 to the planning data originally planned by the system are fed back to a learning device 44 as part of a feedback loop 42, which analyzes the changes to the original planning data and can also determine whether these or similar correction entries occur more frequently or only once or rarely. Taking into account certain learning specifications, such as the weighting of certain corrections according to certain criteria (frequency, sources for certain corrections, diversity of correction sources for similar corrections, etc.), the learning device changes the stored patterns and rules at least tendentially, if necessary repeatedly, until no more manual corrections occur in a given scenario or, for example, corrections are statistically equally frequent in different directions.

According to the corrected planning data 38 and its graphical representation 39, tissue treatment 40 of the retina is performed on the patient.

After treatment 40, the success of the treatment is monitored 41, for example by or with the aid of a monitoring device. This success control can include a new image of the retina, for example in the form of newly acquired image data, but in principle any type of data collection by the surgeon and/or the patient. For example, the surgeon and/or the patient may answer standard questions, and the answers may be entered in the form of data and passed to the processing device as an implementation of the feedback loop 43.

The data determined by the monitoring enables a final success control for the individual case and can also be used to improve the assignment algorithm or the patterns and rules of the assignment device 33 as part of a learning process controlled by the learning device 44.

The processing device 20 can be present locally at the laser treatment site or can also be designed as a central device, which is located remotely from the other elements shown in FIGS. 2 and 3 and carries out data processing and determination of planning datasets for a plurality of treatment stations with treatment lasers. In the course of cooperation with a large number of treatment stations, the processing device receives a large amount of training data if it is set up as a learning device, so that the assignment algorithm for the planning data can be improved quickly.

The input or transfer of personal data can generally take place at the laser treatment site or also non-locally, for example in a central office or also in a distributed computer system. Likewise, corrective input from a surgeon or other expert, as well as monitoring, if it is performed without the direct collection of retinal measurement data, can take place on site at the treatment facility in the immediate vicinity of the patient or remotely at a control center or via a distributed computer system.

FIG. 4 shows an exemplary flow chart for a method according to the invention for creating a planning file for laser treatment of the retina of an eye.

In a first step 45, all previously known data, i.e. the patient's personal data, such as a diagnosis, a treatment indication and, if applicable, a treatment status, if pre-treatment has already taken place, and other known physiological data are retrieved and imported into the system. This can be done by transferring data in electronic form or by manual input. In a second step 46, a camera or other imaging device is used to create a current image of the patient's retina, i.e. a data representation of location-dependent data of the retina. This data is processed in a third step 42 in such a way that structures and/or objects are recognized, and the data is converted into structured data. In the next, fourth step 48, a planning dataset for a retinal treatment is created, taking into account the personal data and the structured image data, using existing, stored templates in the form of patterns and rules. The planning data can also already contain a template for a report on the specifications and the implementation and, if applicable, the success of the treatment. If necessary, the planning data is then corrected by a surgeon in a subsequent step 49. In one embodiment of the method, the corrected planning data can be sent in a step 50 to a training process 54, which influences the process 48 for generating planning data according to the existing patterns and rules and, for this purpose, collects and evaluates experience data within the training management 53 and, if necessary, releases it as training data and also weights it.

After treatment of the retina according to the applicable planning data, which is not shown in FIG. 4 itself, monitoring 51 is carried out in the form of a success evaluation or success acquisition, which can be carried out automatically or manually on the basis of measurement data and/or the assessment of a person. In a further step 52, the performance evaluation can be introduced into the training process 54 in order to improve the planning data generation process 48.

The process according to the invention can be used to continuously improve the method for generating planning files/planning data for laser treatment of the retina.

By using an automated system improved in this way, the various known treatments can be planned in a reliable, comprehensible and objective manner with little effort.

1. A system for generating planning datasets for tissue treatment of a retina of an eye of a patient using of a laser, the system comprising:
a camera configured to continuously acquire images of the retina;
an image processing configured to generate and provide current structured image data of the retina from images captured by the camera;
a personal data acquisition device configured to at least one of acquire or provide personal data of patient; and
a processing device configured to assign one or more planning datasets for the tissue treatment to the current structured image data of the retina and to the personal data of the patient, with specifications for the assignment of the one or more planning datasets to the current structured image data and the personal data of the patient being stored in the processing device, wherein the specifications include one or more patterns or rules.

2. The system according to claim 1, wherein a patient category selected from a plurality of predefined patient categories is assigned to the patient based at least in part on the personal data or the acquired data, and the specifications and wherein the personal data are determined or influenced by the assigned patient category.

3. The system according to claim 1, wherein the personal data acquisition device is configured to acquire and provide at least one of: i) diagnostic data, ii) indication data, iii) data of a pre-treatment carried out, iv) previously known physiological data of the patient, or v) one or more existing images of the retina of the patient stored outside the system.

4. The system according to claim 1, further comprising:
a measurement data acquisition device including one or more sensor devices for acquiring and providing physiological data of the patient, wherein a first portion of the physiological data which depends on a respective location on the retina, wherein a second portion of the physiological data does not depend on a location on the retina, and wherein the processing device is configured to take the acquired or provided physiological data of the patient into account when assigning planning datasets.

5. The system according to claim 1, further comprising:
a display module to display a graphical representation of the one or more planning datasets within an image of the retina currently captured by the camera.

6. The system according to claim 5, further comprising:
a tracking device configured to cause the display of the one or more planning datasets to be tracked when the image of the retina currently captured by the camera is displaced.

7. The system according to claim 4, wherein the processing device has a storage device configured to store the personal data of the patient, the one or more planning datasets assigned to the patient, the physiological data, or one or more manual modifications made to the one or more planning datasets.

8. The system according to claim 4, wherein the processing device has a storage device which is configured to store at least one of the personal data of the patient, the current structured image data, or images of the patient's retina generated with the one or more planning datasets assigned to the patient, and to store the physiological data and one or more manual modifications made to the one or more planning datasets.

9. The system according to claim 4, further comprising:
an inspection device configured to, after a treatment of the retina is performed, at least one of acquire or provide data on an effect of the treatment.

10. The system according to claim 9, wherein the processing device is configured as a self-learning device, wherein to assign the one or more planning datasets, includes assigning one or more target values for measurement data to the personal data to and at least one of the structured image data, or the measured physiological data of a patient, and wherein the processing device is trained by having specified for, in each case for one patient, at least data from:
the personal data;
the structured image data;
the measured physiological data;
the assigned planning data;
the target values for measurement data;
one or more manual changes to planning data; and
the data on the effect of the treatment.

11. The system according to claim 1, wherein the one or more planning datasets include planning data of at least one of, a laser parameter, location data for laser treatment, one or more treatment patterns, location data for one or more regions excluded from laser treatment, or one or more target values for measurement data.

12. A device for generating one or more planning datasets for tissue treatment of a retina of an eye, the device comprising:
a camera, configured to continuously acquire images of the retina;
an image processing device configured to generate and provide current structured image data of the retina from the of the retina captured by the camera;
a personal data acquisition device continue to at least one of acquire or provide personal data of a patient;
a processing device configured to assign the one or more planning datasets to the current structured image data of the retina and to the personal data of the data, wherein one or more specifications for the assignment of planning data to structured image data and the personal data of the patient are stored in the processing device; and
a modification device configured to enable manual modification of a planning dataset.

13. (canceled)

14. A method for generating one or more planning datasets for tissue treatment of a retina of an eye of a patient, the method comprising:
capturing at least one image of the retina using a camera;
generating or providing, using an image processing device, current structured image data of the retina from the at least one image captured by the camera;
acquiring or providing, using a personal data acquisition device, personal data of the patient; and
assigning, using a processing device, the one or more planning datasets to the current structured image data of the retina and the personal data of the patient, based at least in part on one or more specifications stored in the processing device, wherein the one or more specifications include one or more patterns or rules.

15. The method according to claim 14, wherein the assigned one or more planning datasets are changed manually after assignment by the processing device.

16. The method according to claim 14, further comprising:
acquiring, using a measurement data acquisition device, physiological data of the patient, wherein the processing device uses the physiological data when assigning the one or more planning datasets.

17. The method according to claim 14, wherein the processing device is designed as a self-learning device and the processing device is trained using at least one of the personal data of the patient, the structured image data, the assigned one or more planning datasets, manual modifications of the one or more planning datasets, or data on an effect of the treatment acquired after planned treatment has been carried out for each patient of a plurality of patients.

18. The method according to claim 14, wherein planning data of at least one of a laser parameter, location data for laser treatment, one or more treatment patterns, location data for one or more regions excluded from laser treatment, or one or more target values for measurement data are determined as the one or more planning datasets.

19. The method according to claim 14, further comprising:
implementing a treatment using the one or more planning datasets in a treatment device using a treatment laser.

20. The system according to claim 4, further comprising:
a modification device configured to allow manual modification of the one or more planning datasets.

21. The device according to claim 12, wherein the one or more planning datasets include planning data of at least one of: a laser parameter, location data for laser treatment, one or more treatment patterns, location data for one or more regions excluded from laser treatment, or one or more target values for measurement data.
